(19) United States
(12) Patent Application Publication
(10) Pub. No.: US 2025/0187876 A1
(43) Pub. Date: Jun. 12, 2025

(54) LIFTING DEVICES

(71) Applicant: Chinabridge (Shenzhen) Medical Technology Co., Ltd., Shenzhen (CN)

(72) Inventor: Yijiang Li, Shenzhen (CN)

(21) Appl. No.: 18/926,069

(22) Filed: Oct. 24, 2024

(30) Foreign Application Priority Data
Oct. 18, 2023 (CN) 202311365770.1

(51) Int. Cl. B66B 7/02; A61M 1/16

(52) U.S. Cl. CPC B66B 7/02; A61M 1/1698; A61M 2209/084

(57) ABSTRACT

Examples of the present application provide an ECMO system lifting device, including: a support device, a lifting device, a first guide device, and a second guide device. The support device includes a base and a vertical column. The lifting device includes a support and a lifting rod. The first guide device includes a first adjustment mechanism and a first guide wheel set, the first guide wheel set includes a first guide wheel and a second guide wheel, and the first adjustment mechanism can cause the first guide wheel and the second guide wheel to move relative to each other in a first direction, so that circumferential surfaces of the first guide wheel and the second guide wheel each abut against the vertical column. The second guide device includes a second adjustment mechanism and a second guide wheel set, and the second guide wheel set includes a third guide wheel and a fourth guide wheel, so that circumferential surfaces of the third guide wheel and the fourth guide wheel each abut against the vertical column. Every two of the first direction, the second direction, and a length direction of the vertical column are perpendicular to each other. The ECMO system lifting device according to the present invention can be implemented to solve problems in the prior art relating to steady and convenient lifting and lowering of ECMO systems.

### TECHNICAL FIELD

Examples of the present application relate to the field of medical instruments, and in particular, to an ECMO system lifting device.

### BACKGROUND

Extracorporeal membrane oxygenation (ECMO) is primarily used to provide continuous extracorporeal respiration and circulation for patients with severe cardiopulmonary function failures to sustain the patients' lives. The core components of an ECMO system are a membrane lung (artificial lung) and a blood pump (artificial heart). During operation, the system needs to draw a patient's blood out of the body through a conduit, and then use the blood pump to pass the blood through the membrane lung for oxygenation before returning the blood to the patient's body. In clinical use, since the blood pump needs to draw out the patient's blood, it is usually necessary to affix the blood pump at a position as low as possible relative to the patient's heart, to ensure sufficient and smooth blood drainage. However, this makes it inconvenient for medical staff to operate the blood pump and the membrane lung during priming. Therefore, the ECMO system requires a lifting device to adjust the heights of the membrane lung and the blood pump, thereby enhancing the convenience of operation during the application of the ECMO system.

Normally, when a patient is lying on a bed, the patient's heart is at least 0.5 meters above a bottom surface, so to ensure sufficient negative pressure drainage, it is desirable that the lifting height be at least 0.5 meters.

Moreover, a patient on an ECMO system requires frequent turning by medical staff due to a prolonged bedridden period, which leads to a need for a very long conduit for connecting the blood pump and the membrane lung to the patient. However, extending the conduit increases circulatory resistance and priming volume, which is highly detrimental to patient treatment. Therefore, it is also necessary to adjust the heights of the membrane lung and the blood pump. During turning operations, the membrane lung and the blood pump can be adjusted to higher positions, and restored to lower positions for work once the turning operations are complete. For this purpose, a convenient and safe lifting device is needed, to ensure that the medical staff can operate the lifting device reliably without dragging the conduit and harming the patient due to abnormal lifting or lowering of the lifting device caused by accidental operations or single-point failures.

In addition, since the blood pump transmits torque by magnetic coupling, a fixing device for the blood pump needs to be steady and cannot experience vigorous shaking during operation. Otherwise, a rotor inside the blood pump can be easily damaged by impact or the service life of the rotor may be reduced. Therefore, the lifting device must operate steadily, without producing vigorous shaking.

### SUMMARY

To solve or alleviate problems in the prior art relating to the steady and convenient lifting and lowering of ECMO systems, the present invention provides an ECMO system lifting device, comprising:

A support device, wherein the support device comprises a base and a vertical column, the base being disposed at the bottom of the vertical column, the vertical column being hollow, to form a cavity, and a surface of the vertical column being provided with an opening arranged along an axial direction of the vertical column, the opening communicating with the cavity;

A lifting device, wherein the lifting device comprises a support and a lifting rod, the lifting rod being disposed inside the cavity, the support extending into the opening and being fixedly connected to the lifting rod, and the support being lifted and lowered following lifting and lowering of the lifting rod;

A first guide device, wherein the first guide device is disposed inside the cavity and connected to the support, the first guide device comprises a first adjustment mechanism and a first guide wheel set, the first guide wheel set comprises a first guide wheel and a second guide wheel, and the first adjustment mechanism can cause the first guide wheel and the second guide wheel to move relative to each other in a first direction, so that circumferential surfaces of the first guide wheel and the second guide wheel each abut against the vertical column; and

A second guide device, wherein the second guide device is disposed inside the cavity and connected to the support, the second guide device comprises a second adjustment mechanism and a second guide wheel set, the second guide wheel set comprises a third guide wheel and a fourth guide wheel, and the second adjustment mechanism can cause the third guide wheel and the fourth guide wheel to move relative to each other in a second direction, so that circumferential surfaces of the third guide wheel and the fourth guide wheel each abut against the vertical column;

Every two of the first direction, the second direction, and a length direction of the vertical column are perpendicular to each other.

As a preferred example of the present application, the first adjustment mechanism and/or the second adjustment mechanism comprises an elastic member, and the elastic member causes the first guide wheel and the second guide wheel to move relative to each other in the first direction, and/or causes the third guide wheel and the fourth guide wheel to move relative to each other in the second direction.

As a preferred example of the present application, the opening is traversable along the first direction, the first adjustment mechanism comprises a first adjustment frame and a first screw, the first guide wheel is disposed on the support and always abuts against the vertical column, the second guide wheel is disposed on the first adjustment frame, the first adjustment frame is slidably connected to the support by means of the first screw, and rotating the first screw can change a distance in the first direction between the first adjustment frame and the support;

The second adjustment mechanism comprises a second adjustment frame, a second screw, and a slider sleeved on the second screw, the second adjustment frame is slidably connected to the support, the second adjustment frame is provided with the third guide wheel, the support is provided with the fourth guide wheel, which always abuts against the vertical column, the slider is provided with a first inclined surface, the adjustment frame is provided with a second inclined surface corresponding to the first inclined surface, and rotating the second screw can cause the slider to move and push the second adjustment frame to move along the second direction; and

At least one end of each of the first screw and the second screw is exposed from the opening.

As a preferred example of the present application, a plurality of lifting rods are provided, and the plurality of lifting rods are stacked, so that a total lifting stroke of the ECMO system lifting device is the sum of respective strokes of the plurality of lifting rods.

As a preferred example of the present application, the lifting device further comprises a fixing frame, the fixing frame being used to fixedly connect two adjacent lifting rods.

As a preferred example of the present application, a safety brake device is further comprised, wherein the safety brake device is disposed inside the cavity and fixedly connected to the support, and the safety brake device can abut against the vertical column to prevent lifting and lowering of the lifting rod.

As a preferred example of the present application, a control device is further comprised, wherein the control device can unlock the safety brake device to separate the safety brake device from the vertical column.

As a preferred example of the present application, the control device can be further linked to the lifting rod, and the lifting rod starts to extend when the control device controls the safety brake device to be separated from the vertical column.

As a preferred example of the present application, the control device is fixedly disposed on the vertical column, the control device comprises a grip member, and the lifting rod and the safety brake device are simultaneously linked by toggling the grip member.

As a preferred example of the present application, a carrier platform is further comprised, the carrier platform being disposed outside the vertical column and fixedly connected to the support.

Compared with existing technologies, the ECMO system lifting device according to the present invention has a first guide device and a second guide device, the lifting device, the first guide device and the second guide device all being disposed inside the cavity of a vertical column, and only the surface of the vertical column being provided with an opening arranged along the axial direction of the vertical column. Therefore, the ECMO system lifting device according to the present invention features safety, and prevents human injuries caused by splashing parts resulted from failures of the lifting device, the first guide device, and the second guide device. Further, since every two of the first guide device, the second guide device, and the length direction of the vertical column are arranged perpendicular to each other, the first guide device and the second guide device can ensure that the support is kept steady when being pushed or lifted and lowered by the lifting rod. The lifting rod automatically extends and retracts, thereby avoiding excessive operations by medical staff. Therefore, the ECMO system lifting device according to the present invention can be implemented to solve problems in the prior art relating to steady and convenient lifting and lowering of the ECMO system in existing technologies.

### DESCRIPTION OF THE DRAWINGS

The accompanying drawings described herein are used to provide further understanding of the present application, and constitute a part of the present application. The illustrative examples of the present application and the descriptions thereof are used to explain the present application and do not constitute an improper limitation on the present application. Some specific examples of the present application will be described in detail below by way of example but not limitation with reference to the accompanying drawings. The same reference numerals in the drawings represent the same or similar components or portions. A person skilled in the art should understand that the drawings are not necessarily drawn to scale. In the drawings:
support device—10; base—110; vertical column—120; cavity—1110; opening—1120; lifting device—20; support—210; lifting rod—220; first guide device—30; first guide wheel set—320; first guide wheel—3210; second guide wheel—3220; second guide device—40; second guide frame—410; second guide wheel set—430; third guide wheel—4310; fourth guide wheel—4320; first adjustment frame—3110; first screw—3120; second adjustment frame—4210; second screw—4220; slider—4230; fixing frame—230; safety brake device—50; control device—60; grip member—610; carrier platform—70; elastic member—80.

FIG. 1 shows a schematic diagram of an overall structure of an ECMO system lifting device according to an example of the present invention;

FIG. 2 shows a schematic exploded structural diagram of an ECMO system lifting device according to an example of the present invention;

FIG. 3 shows a rear view of a structure of an ECMO system lifting device with a support device removed according to an example of the present invention;

FIG. 4 shows a rear view of a structure of an ECMO system lifting device with a support device removed according to another example of the present invention;

FIG. 5 shows a local enlarged view of part A in FIG. 4;

FIG. 6 shows a schematic cross-sectional view of an ECMO system lifting device at a first guide device according to an example of the present invention;

FIG. 7 shows a schematic cross-sectional view of an ECMO system lifting device at a second guide device according to an example of the present invention;

FIG. 8 shows a schematic structural diagram of lifting rods in a first assembly state according to an example of the present invention;

FIG. 9 shows a schematic structural diagram of lifting rods in a second assembly state according to an example of the present invention; and

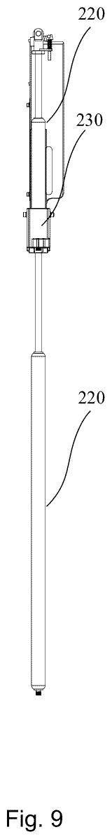

FIG. 10 shows a schematic structural diagram of lifting rods in a third assembly state according to an example of the present invention.

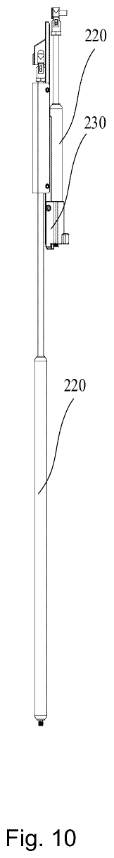

### DESCRIPTION OF EMBODIMENTS

To enable a person skilled in the art to better understand the solutions of the present application, the technical solutions in the examples of the present application are clearly and thoroughly described below with reference to the accompanying drawings in the examples of the present application. Clearly, the described examples are merely some rather than all of the examples of the present application. All other examples obtained by a person of ordinary skill in the art based on the examples of the present application without creative efforts shall fall within the scope of protection of the present application.

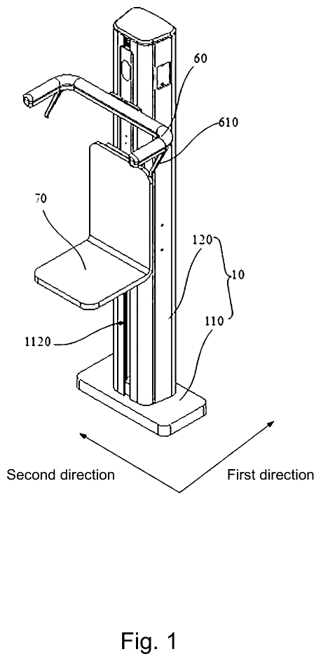

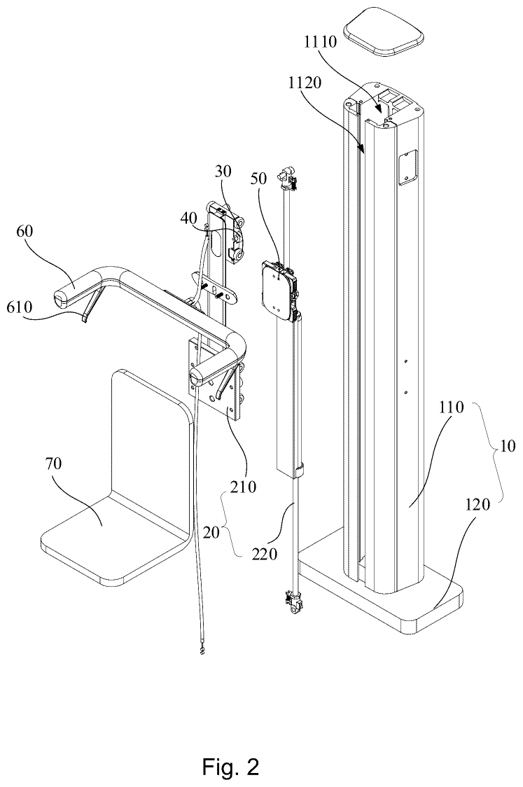

To solve problems in the prior art relating to steady and convenient lifting and lowering of ECMO systems, referring to FIG. 1 to FIG. 3, the present invention provides an ECMO system lifting device 20, including:

A support device 10, wherein the support device 10 includes a base 110 and a vertical column 120, the base 110 being disposed at the bottom of the vertical column 120, the vertical column 120 being hollow, to form a cavity 1110, and a surface of the vertical column 120 being provided with an opening 1120 arranged along an axial direction of the vertical column 120, the opening 1120 communicating with the cavity 1110;

A lifting device 20, wherein the lifting device 20 includes a support 210 and a lifting rod 220, the lifting rod 220 being disposed inside the cavity 1110, the support 210 extending into the opening 1120 and being fixedly connected to the lifting rod 220, and the support 210 being lifted and lowered following lifting and lowering of the lifting rod 220;

A first guide device 30, wherein the first guide device is disposed inside the cavity 1110 and connected to the support 210, the first guide device 30 includes a first adjustment mechanism and a first guide wheel set (not shown), the first guide wheel set (not shown) includes a first guide wheel 3210 and a second guide wheel 3220, and the first adjustment mechanism (not shown) can cause the first guide wheel 3210 and the second guide wheel 3220 to move relative to each other in a first direction, so that circumferential surfaces of the first guide wheel 3210 and the second guide wheel 3220 each abut against the vertical column 120; and

A second guide device 40, wherein the second guide device is disposed inside the cavity 1110 and connected to the support 210, the second guide device 40 includes a second adjustment mechanism and a second guide wheel set (not shown), the second guide wheel set (not shown) includes a third guide wheel 4310 and a fourth guide wheel 4320, and the second adjustment mechanism (not shown) can cause the third guide wheel 4310 and the fourth guide wheel 4320 to move relative to each other in a second direction, so that circumferential surfaces of the third guide wheel 4310 and the fourth guide wheel 4320 each abut against the vertical column 120;

Every two of the first direction, the second direction, and a length direction of the vertical column 120 are perpendicular to each other.

Compared with existing technologies, the ECMO system lifting device 20 according to the present invention has a first guide device 30 and a second guide device 40, the lifting device 20, the first guide device 30 and the second guide device 40 all being disposed inside the cavity 1110 of a vertical column 120, and only the surface of the vertical column 120 being provided with an opening 1120 arranged along the axial direction of the vertical column 120. Therefore, the ECMO system lifting device 20 according to the present invention features safety, and prevents from human injuries caused by flying parts resulting from failures of the lifting device 20, the first guide device 30 and the second guide device 40. Further, since every two of the first guide device 30, the second guide device 40, and the length direction of the vertical column 120 are arranged perpendicular to each other, the first guide device 30 and the second guide device 40 can ensure that the support 210 is kept steady when being pushed or lifted and lowered by the lifting rod 220. The lifting rod 220 automatically extends and retracts, thereby avoiding excessive operations by medical staff. Therefore, the ECMO system lifting device 20 according to the present invention can be implemented to solve problems in the prior art relating to steady and convenient lifting and lowering of ECMO systems.

Generally, the support device 10 includes the base 110 and the vertical column 120, wherein the vertical column 120 is usually integrally formed from a metallic material. For specifications of the vertical column 120, consistent dimensional parameters are often difficult to achieve due to mass production considerations. The ECMO system lifting device 20 is a delicate and important medical instrument, and needs to maintain steady lifting and lowering. Therefore, the ECMO system lifting device is provided with guide devices to ensure steadiness during lifting and lowering. The first guide device 30 and the second guide device 40 each ensure steadiness of the lifting device 20 in one plane, and a combined effect of the two guide devices enables the lifting device 20 to move only along the length direction of the vertical column 120.

In this example, the support 210 mainly provides two functions. In one aspect, the support is used to mount an external medical apparatus to implement lifting and lowering of the external medical apparatus. In another aspect, the support 210 is used to mount the first guide device 30 and the second guide device 40.

It should be noted that, in the first guide device 30, there may be a plurality of first guide wheel sets (not shown), which are arranged along the length direction of the vertical column 120. The first guide wheel set (not shown) includes the first guide wheel 3210 and the second guide wheel 3220, and the first adjustment mechanism can cause the first guide wheel 3210 and the second guide wheel 3220 to move relative to each other in the first direction. In other words, a distance between the first guide wheel 3210 and the second guide wheel 3220 is adjusted, so that the first guide wheel 3210 and the second guide wheel 3220 each abut against an inner wall of the vertical column 120, ultimately limiting the lifting device 20 in the first direction.

In the second guide device 40, there may also be a plurality of second guide wheel sets (not shown), which are arranged along the length direction of the vertical column 120. The second guide wheel set (not shown) includes the third guide wheel 4310 and the fourth guide wheel 4320, and the second adjustment mechanism can cause the third guide wheel 4310 and the fourth guide wheel 4320 to move relative to each other in the second direction. In other words, a distance between the third guide wheel 4310 and the fourth guide wheel 4320 is adjusted, so that the third guide wheel 4310 and the fourth guide wheel 4320 each abut against an inner wall of the vertical column 120, ultimately limiting the lifting device 20 in the second direction.

The first guide wheel set (not shown) and the second guide wheel set (not shown) are usually spaced apart. In some cases, the first guide device 30 and the second guide device 40 may alternatively be modularized to facilitate mass production and assembly.

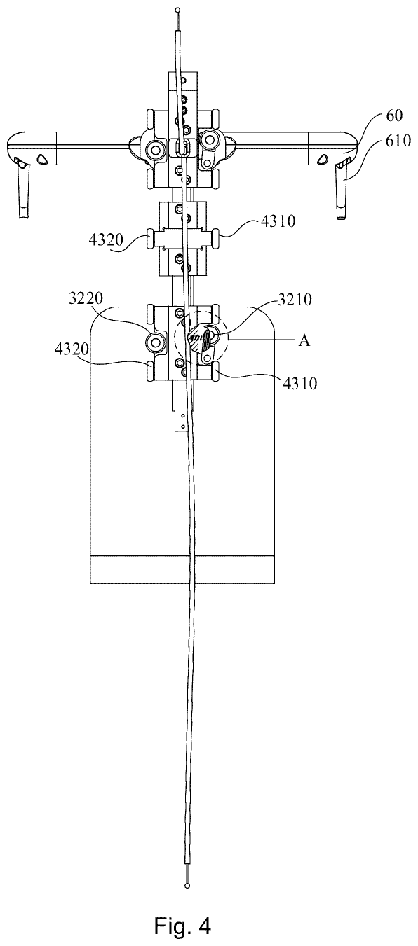

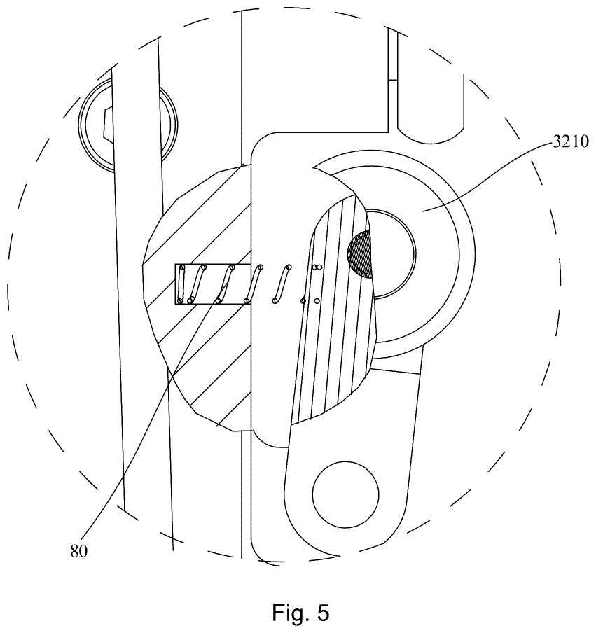

In a preferred example, referring to FIG. 4 and FIG. 5, the first adjustment mechanism and/or the second adjustment mechanism includes an elastic member 80, and the elastic member 80 causes the first guide wheel 3210 and the second guide wheel 3220 to move relative to each other in the first direction and/or causes the third guide wheel 4310 and the fourth guide wheel 4320 to move relative to each other in the second direction.

The first adjustment mechanism (not shown) and the second adjustment mechanism (not shown) may also adjust the distance between the guide wheels in another manner. The relative movement between the first guide wheel 3210 and the second guide wheel 3220 may be such that the first guide wheel 3210 and the second guide wheel 3220 move simultaneously, or one of the two guide wheels is stationary while the other moves. Accordingly, the third guide wheel 4310 and the fourth guide wheel 4320 have the same motion relationship.

In this example, the elastic member 80 is generally disposed between the first guide wheel 3210 and the second guide wheel 3220, or between the third guide wheel 4310 and the fourth guide wheel 4320, or both simultaneously. The elastic member is preferably a spring, which expands to cause the two connected guide wheels to abut against the inner wall of the vertical column 120.

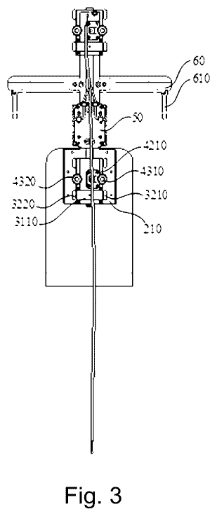

In a preferred example, referring to FIG. 3 to FIG. 5, the opening 1120 is traversable along the first direction, the first adjustment mechanism includes a first adjustment frame 3110 and a first screw 3120, the first guide wheel 3210 is disposed on the support 210 and always abuts against the vertical column 120, the second guide wheel 3220 is disposed on the first adjustment frame 3110, the first adjustment frame 3110 is slidably connected to the support 210 by means of the first screw 3120, and rotating the first screw 3120 can change a distance in the first direction between the first adjustment frame 3110 and the support 210;

The second adjustment mechanism includes a second adjustment frame 4210, a second screw 4220, and a slider 4230 sleeved on the second screw 4220, the second adjustment frame 4210 is slidably connected to the support 210, the second adjustment frame 4210 is provided with the third guide wheel 4310, the support 210 is provided with the fourth guide wheel 4320, which always abuts against the vertical column 120, the slider 4230 is provided with a first inclined surface, the adjustment frame is provided with a second inclined surface corresponding to the first inclined surface, and rotating the second screw 4220 can cause the slider 4230 to move and push the second adjustment frame 4210 to move along the second direction; and

At least one end of each of the first screw 3120 and the second screw 4220 is exposed from the opening 1120.

In this example, the first adjustment mechanism is used to adjust the distance between the first guide wheel 3210 and the second guide wheel 3220, and the second adjustment mechanism is used to adjust the distance between the third guide wheel 4310 and the fourth guide wheel 4320. In general, before the ECMO system lifting device 20 according to the present invention is delivered from the factory, commissioning is performed once, so that the ECMO system lifting device 20 according to the present invention can implement steady lifting and lowering.

The first adjustment mechanism (not shown) includes the first adjustment frame 3110 and the first screw 3120, and the second guide wheel 3220 is not directly connected to the support 210 but is connected to the support 210 through the first adjustment frame 3110. Therefore, the first guide wheel 3210 and the second guide wheel 3220 are movable in the first direction, so that the circumferential surfaces of the first guide wheel 3210 and the second guide wheel 3220 both abut against the inner wall of the vertical column 120. The first screw 3120, the first adjustment frame 3110, and the support 210 are equivalent to one lead screw structure. The opening 1120 is traversable along the first direction. This ensures that an adjustment end of the first screw 3120 can be exposed from the opening 1120 to facilitate operations by production staff.

The second adjustment mechanism (not shown) includes the second adjustment frame 4210, the second screw 4220, and the slider 4230 sleeved on the second screw 4220. In this case, the second screw 4220, the slider 4230, and the support 210 are also equivalent to one lead screw structure. However, such an implementation only allows the second screw 4220 to be disposed along the second direction. Consequently, another opening 1120 needs to be started on the vertical column 120.

With the second adjustment frame 4210 and the slider 4230, a directional change is achieved, which enables the second screw 4220 to be disposed along the first direction while ensuring that the distance in the second direction between the third guide wheel 4310 and the fourth guide wheel 4320 can be changed.

Specifically, the slider 4230 is provided with the first inclined surface, and the second adjustment frame 4210 is provided with the second inclined surface. The first inclined surface and the second inclined surface abut against each other, and can slide relative to each other. When the second screw 4220 is rotated, the slider 4230 moves along a length direction of the second screw 4220, and the first inclined surface presses against the second inclined surface. As the second adjustment frame 4210 cannot move in the first direction, when pressed against by the first inclined surface, the second adjustment frame 4210 drives the third guide wheel 4310 to move in the second direction.

In general, before commissioning upon delivery from the factory, the fourth guide wheel 4320 and the first guide wheel 3210 are pre-adjusted to abut against the inner wall of the vertical column 120.

In a preferred example, a plurality of lifting rods 220 are provided, and the plurality of lifting rods 220 are stacked, so that a total lifting stroke of the ECMO system lifting device 20 is the sum of respective strokes of the plurality of lifting rods 220.

In this example, the lifting rod 220 is generally a standard member, and in some cases, a single lifting rod 220 cannot achieve a sufficient lifting distance. Therefore, a plurality of lifting rods 220 need to be stacked to ensure that the ECMO system lifting device 20 according to the present invention can have a sufficient lifting height.

In a preferred example, the lifting device 20 further includes a fixing frame 230, and the fixing frame 230 is configured to fixedly connect two adjacent lifting rods 220.

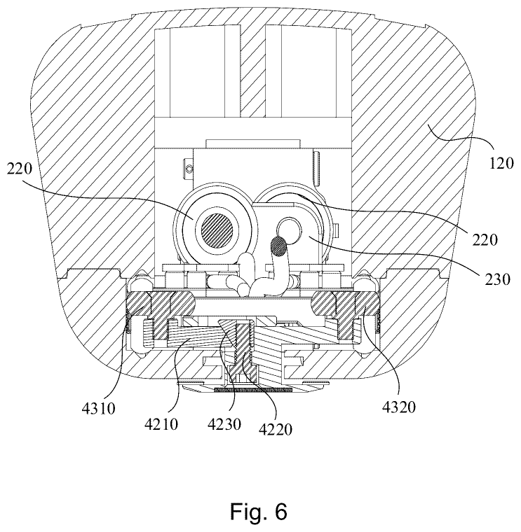

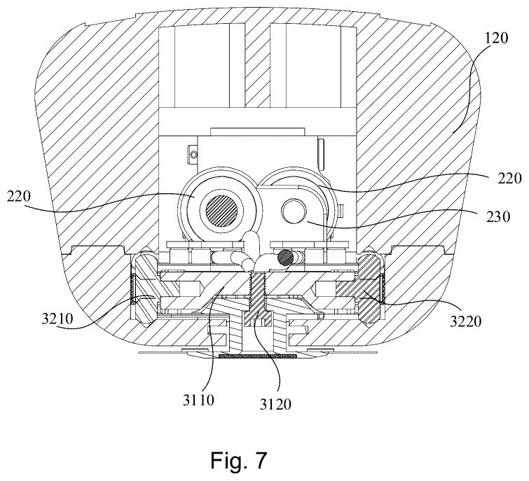

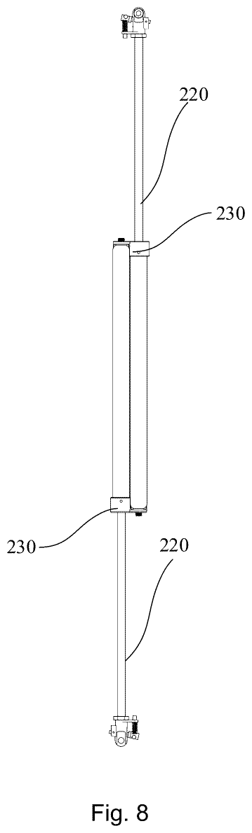

In this example, the fixing frame 230 is configured to connect two adjacent lifting rods 220. Referring to FIG. 6, the lifting rod 220 generally has a fixed rod and a telescoping rod. The fixed rods of two lifting rods 220 are fixedly connected to each other. Referring to FIG. 7, the fixed rod and the telescoping rod are connected end to end. Referring to FIG. 8, through adaptation by the fixing frame 230, the fixed rod of one lifting rod 220 is connected in parallel to the telescoping rod of another lifting rod 220, thereby preventing the lifting device 20 from failing to reach a small height.

In a preferred example, a safety brake device 50 is further included, the safety brake device 50 being disposed inside the cavity 1110 and fixedly connected to the support 210, and the safety brake device 50 being able to abut against the vertical column 120 to prevent extension and retraction of the lifting rod 220.

The safety brake device 50 is provided to ensure safety, so as to prevent a medical accident caused by a performance failure of the lifting rod 220 during treatment. In general, the safety brake device 50 is fixedly mounted on the lifting device 20 and always prevents the lifting rod 220 from lifting or lowering.

In a preferred example, a control device 60 is further included, the control device 60 being able to unlock the safety brake device 50 so as to separate the safety brake device 50 from the vertical column 120.

When height adjustment is required, the safety brake device 50 needs to be unlocked, and the control device 60 can separate the safety brake device 50 from the vertical column 120. The control device 60 may be electrically or mechanically controlled, provided that the control device can control the safety brake device 50 to brake or unlock.

In a preferred example, the control device 60 can be further linked to a pneumatic lifting rod, and the pneumatic lifting rod starts to extend when the control device 60 controls the safety brake device 50 to be separated from the vertical column 120.

The pneumatic lifting rod is a common lifting device 20. When the safety brake device 50 is separated from the vertical column 120, the pneumatic lifting rod can automatically extend and retract. The principle of the pneumatic lifting rod is equivalent to that of an office chair. In the presence of an ECMO system, the pneumatic lifting rod retracts under the weight of the ECMO system, and in the absence of an ECMO system, the pneumatic lifting rod extends.

In a preferred example, the control device 60 is fixedly disposed on the vertical column 120, the control device 60 includes a grip member 610, and the pneumatic lifting rod and the safety brake device 50 are simultaneously linked by toggling the grip member 610.

In this example, the grip member 610 is simultaneously linked to the pneumatic lifting rod and the safety brake device 50. Therefore, after the grip member 610 is toggled, the safety brake device 50 is directly unlocked, and the pneumatic lifting rod starts to work.

In a preferred example, a carrier platform 70 is further included, and the carrier platform 70 is disposed outside the vertical column 120 and fixedly connected to the support 210.

The carrier platform 70 is mainly used to carry a medical instrument, providing a position for mounting the medical instrument. Specifically, the carrier platform 70 is mainly used to carry a membrane lung and a blood pump of an ECMO system. The membrane lung and the blood pump are important components of the ECMO system. The membrane lung is primarily used to supply oxygen to the blood, and the blood pump is used to cause the blood to flow, which is equivalent to cardiac pacing.

A position of the blood pump is equivalent to a position of the heart. Therefore, the height of the blood pump determines a blood pressure level to some extent. The carrier platform 70 is lifted and lowered to adjust the height of the blood pump, ultimately achieving a blood pressure regulation effect.

In the foregoing description, the first direction is generally perpendicular to a plane in which the opening 1120 is located, and the second direction is generally parallel to the plane in which the opening 1120 is located.

Finally, it should be noted that the foregoing examples are only used to illustrate the technical solutions of the present application, rather than constitute limitations thereto. Although the present application is described in detail with reference to the foregoing examples, it should be understood by a person of ordinary skill in the art that one may still modify the technical solutions described in the foregoing examples or equivalently replace some or all technical features therein; and these modifications or replacements do not make the essence of the corresponding technical solutions depart from the scope of the technical solutions of various examples of the present application.

1. An ECMO system lifting device, characterized by comprising:
a support device, wherein the support device comprises a base and a vertical column, the base being disposed at the bottom of the vertical column, the vertical column being hollow, to form a cavity, and a surface of the vertical column being provided with an opening arranged along an axial direction of the vertical column, the opening communicating with the cavity;
a lifting device, wherein the lifting device comprises a support and a lifting rod, the lifting rod being disposed inside the cavity, the support extending into the opening and being fixedly connected to the lifting rod, and the support being lifted and lowered following lifting and lowering of the lifting rod;
a first guide device, wherein the first guide device is disposed inside the cavity and is connected to the support, the first guide device comprises a first adjustment mechanism and a first guide wheel set, the first guide wheel set comprises a first guide wheel and a second guide wheel, and the first adjustment mechanism can cause the first guide wheel and the second guide wheel to move relative to each other in a first direction, so that circumferential surfaces of the first guide wheel and the second guide wheel each abut against the vertical column; and
a second guide device, wherein the second guide device is disposed inside the cavity and is connected to the support, the second guide device comprises a second adjustment mechanism and a second guide wheel set, the second guide wheel set comprises a third guide wheel and a fourth guide wheel, and the second adjustment mechanism can cause the third guide wheel and the fourth guide wheel to move relative to each other in a second direction, so that circumferential surfaces of the third guide wheel and the fourth guide wheel each abut against the vertical column;
every two of the first direction, the second direction, and a length direction of the vertical column are perpendicular to each other.

2. The ECMO system lifting device according to claim 1, wherein the first adjustment mechanism and/or the second adjustment mechanism comprises an elastic member, and the elastic member causes the first guide wheel and the second guide wheel to move relative to each other in the first direction and/or causes the third guide wheel and the fourth guide wheel to move relative to each other in the second direction.

3. The ECMO system lifting device according to claim 1, wherein the opening is traversable along the first direction, the first adjustment mechanism comprises a first adjustment frame and a first screw, the first guide wheel is disposed on the support and always abuts against the vertical column, the second guide wheel is disposed on the first adjustment frame, the first adjustment frame is slidably connected to the support by means of the first screw, and rotating the first screw can change a distance in the first direction between the first adjustment frame and the support;
the second adjustment mechanism comprises a second adjustment frame, a second screw, and a slider sleeved on the second screw, the second adjustment frame is slidably connected to the support, the second adjustment frame is provided with the third guide wheel, the support is provided with the fourth guide wheel, which always abuts against the vertical column, the slider is provided with a first inclined surface, the adjustment frame is provided with a second inclined surface corresponding to the first inclined surface, and rotating the second screw can make the slider move and push the second adjustment frame to move along the second direction; and
at least one end of each of the first screw and the second screw is exposed from the opening.

4. The ECMO system lifting device according to claim 3, wherein a plurality of lifting rods are provided, and the plurality of lifting rods are stacked, so that a total lifting stroke of the ECMO system lifting device is the sum of respective strokes of the plurality of lifting rods.

5. The ECMO system lifting device according to claim 4, wherein the lifting device further comprises a fixing frame, and the fixing frame is configured to fixedly connect two adjacent lifting rods.

6. The ECMO system lifting device according to claim 4, further comprising a safety brake device, wherein the safety brake device is disposed inside the cavity and is fixedly connected to the support, and the safety brake device can abut against the vertical column to prevent lifting and lowering of the lifting rod.

7. The ECMO system lifting device according to claim 5, further comprising a control device, wherein the control device can unlock the safety brake device to separate the safety brake device from the vertical column.

8. The ECMO system lifting device according to claim 7, wherein the control device can further be linked to the lifting rod, and the lifting rod starts to extend when the control device controls the safety brake device to be separated from the vertical column.

9. The ECMO system lifting device according to claim 8, wherein the control device is fixedly disposed on the vertical column, the control device comprises a gripping member, and the lifting rod and the safety brake device are simultaneously linked by toggling the gripping member.

10. The ECMO system lifting device according to claim 1, further comprising a carrier platform, the carrier platform being disposed outside the vertical column and being fixedly connected to the support.
